(19) United States
(12) Patent Application Publication
(10) Pub. No.: US 2011/0208126 A1
(43) Pub. Date: Aug. 25, 2011

(54) LOCKABLE SHIELD ASSEMBLY FOR A SYRINGE

(76) Inventor: John Riemelmoser, South Australia (AU)

(21) Appl. No.: 12/674,422

(22) PCT Filed: Aug. 21, 2007
(86) PCT No.: PCT/AU07/01195

§ 371 Date: May 7, 2011

(51) Int. Cl. A61M 5/32; B23P 17/04

(52) U.S. Cl. 604/198; 29/428

(57) ABSTRACT

A lockable shield assembly for a syringe is described. The lockable shield assembly includes a moveable sleeve adapted to slide longitudinally along a body of the syringe from a retracted position where the needle of the syringe is exposed to an extended position wherein the moveable sleeve covers the needle. The assembly further includes a saddle member for attachment to the body of the syringe with the saddle member including a resilient tongue biased outwardly from the saddle member, wherein the resilient tongue engages with an abutment region located on the inner surface of the moveable sleeve to prevent retraction of the moveable sleeve when it has been moved to the extended position.

### CROSS REFERENCE TO RELATED APPLICATIONS

The present application is a National Stage Application claiming the priority of co-pending PCT Application No. PCT/AU2007/001195 filed Aug. 21, 2007. Applicant claims the benefits of both 35 U.S.C. §119 and 35 U.S.C. §120 as to the PCT application, and the entire disclosure of said application is incorporated herein by reference in its entirety.

### FIELD OF THE INVENTION

The present invention relates to a lockable sliding shield assembly for a syringe to reduce the possibility of needle stick injuries. In one particular form, the present invention relates to a method for manufacturing and assembling a lockable sliding shield for a syringe.

### BACKGROUND OF THE INVENTION

There are many devices that have been developed for the prevention or reduction of the incidence of needle stick injuries to users of syringes and similar products. Many of these devices rely on retracting the syringe body and needle within an outer sleeve by employing a spring activated mechanism.

Whilst in principle this addresses the general problem of how to retract or cover a needle after use by medical personnel there has been strong resistance to the uptake of these devices due to the added costs involved in their manufacture. As an example, the cost of implementing a spring activated retraction mechanism may triple or even quadruple the cost of a syringe due to the added parts required and the complexity of assembly.

In our earlier filed patent application, PCT Publication No. 2006/072135, entitled “A Lockable Shield for a Syringe” there is described a shield for a syringe with a needle that is moveable between a retracted position where the syringe and needle can be used and a shielding position where at least the tip of the needle is covered to prevent accidental needle stick injuries. The shield further includes locking means to prevent rearward movement of the shield once it has been manually actuated. Although this arrangement is far more cost effective than the spring activated retraction mechanisms of the prior art arrangements and still considered quite satisfactory, further developments have been made in order to simplify the manufacture and assembly of this type of shield in order to make it even more cost effective.

It is an object of the present invention to provide an alternative lockable shield assembly that is capable of being manufactured and assembled in a cost effective manner.

### SUMMARY OF THE INVENTION

In a first aspect the present invention accordingly provides a lockable shield assembly for a syringe, the lockable shield assembly including:
a moveable sleeve adapted to slide longitudinally along a body of the syringe from a retracted position wherein a needle of the syringe is exposed to an extended position wherein the moveable sleeve covers the needle of the syringe; and
a saddle member for attachment to the body of the syringe, the saddle member including a resilient tongue biased outwardly from the saddle member, wherein the resilient tongue in use engages with an abutment region located on an inner surface of the moveable sleeve to prevent retraction of the moveable sleeve when it has been moved to the extended position.

Preferably, the moveable sleeve is operable to move to an intermediate position where the sleeve covers the needle of the syringe but is still able to be retracted to expose the needle of the syringe, and wherein the lockable shield assembly further includes indication means to provide a tactile or audible indication to an operator of the lockable shield assembly that the moveable sleeve is in the intermediate position.

Preferably, the indication means forms part of the saddle member.

Preferably, the indication means includes a flexible member that interacts with the abutment region as the abutment region rides over the flexible member to provide the tactile or audible indication.

Preferably, the flexible member is a flexible tab extending upwardly from the saddle member.

Preferably, the resilient tongue faces forwards towards the needle end of the syringe and wherein the flexible tab is located at the base of the resilient tongue.

Preferably, the saddle member includes an abutment portion operable in combination with the resilient tongue to form a catch region to capture the abutment region of the moveable sleeve when the moveable sleeve is in the extended position.

In a second aspect the present invention accordingly provides an assembly method for a lockable shield assembly for a syringe, the lockable shield assembly including a moveable sleeve operable to be moved to an extended position by sliding the moveable sleeve along a body of the syringe and a saddle member including a resilient tongue operable to engage with an abulment region located on the moveable sleeve to prevent retraction of the moveable sleeve once it has been moved to the extended position, the method including the steps of:
inserting the body of the syringe into a first receiving channel formed in the moveable sleeve;
inserting the saddle member into a second receiving channel formed in the moveable sleeve, the second receiving channel connected to the first receiving channel; and
attaching the saddle member to the body of the syringe.

Preferably, the moveable sleeve includes an access region to allow attachment of the saddle member to the body of the syringe.

Preferably, the access region is located at a needle end of the moveable sleeve and is formed as a cutaway region.

Preferably, the resilient tongue frictionally engages with an inner surface of the second receiving channel to resist movement of the saddle member with respect to the saddle member prior to assembly.

Preferably, the saddle member is attached to the body of the syringe by ultrasonic welding.

In a third aspect the present invention accordingly provides a saddle member for a lockable shield assembly for a syringe, the saddle member including:
an attachment region for attachment to a body of the syringe; and
a resilient tongue extending outwardly from the saddle member, the resilient tongue operable to engage with an abutment region located on a moveable sleeve of the lockable shield assembly to prevent retraction of the moveable sleeve once it has been moved to an extended position.

Preferably, the resilient tongue is adapted to frictionally engage with an inner surface of the moveable sleeve to locate the saddle member on insertion of the saddle member into the sleeve for attachment of the saddle member to the body of the syringe on assembly of the lockable shield assembly.

Preferably, the saddle member further includes indication means to provide a tactile or audible indication to an operator of the lockable shield assembly that the moveable sleeve is in an intermediate position where the sleeve covers the needle of the syringe but is still able to be retracted to expose the needle of the syringe.

Preferably, the indication means includes a flexible member that interacts with the abutment region as the abutment region rides over the flexible member to provide the tactile or audible indication.

Preferably, the flexible member is a flexible tab extending upwardly from the saddle member.

Preferably, the resilient tongue faces forwards towards the needle end of the syringe and wherein the flexible tab is located at the base of the resilient tongue.

Preferably, the saddle member includes an abutment portion operable in combination with the resilient tongue to form a catch region to capture the abutment region of the moveable sleeve when the moveable sleeve is in the extended position.

In a fourth aspect the present invention accordingly provides a moveable sleeve for a lockable shield assembly for a syringe, the moveable sleeve adapted to slide longitudinally along a body of the syringe from a retracted position wherein a needle of the syringe is exposed to an extended position wherein the sleeve covers the needle of the syringe, the moveable sleeve further including an abutment region operable to engage with a resilient tongue extending from a saddle member of the lockable shield assembly to prevent retraction of the moveable sleeve once it has been moved to an extended position.

Preferably, the moveable sleeve further includes an access region to allow attachment of the saddle member to the body of the syringe.

Preferably, the access region is located at a needle end of the moveable sleeve and is formed as a cutaway region.

### BRIEF DESCRIPTION OF THE DRAWINGS

A preferred embodiment of the present invention will be discussed with reference to the accompanying drawings wherein:

FIG. 1 is a front perspective view of a lockable shield assembly according to a preferred embodiment of the present invention;

FIG. 2 is a side view of the lockable shield assembly illustrated in FIG. 1;

FIG. 3 is a front end view of the lockable shield assembly illustrated in FIG. 1;

FIGS. 4A-4C depict the operation of the lockable shield assembly illustrated in FIG. 1;

FIG. 5 is a side disassembled view of the components of the lockable shield assembly illustrated in FIG. 1;

FIGS. 6A-6C depict figuratively the steps in assembling the components illustrated in FIG. 5 to provide the lockable shield assembly illustrated in FIG. 1.

In the following description, like reference characters designate like or corresponding parts throughout the several views of the drawings.

### DESCRIPTION OF PREFERRED EMBODIMENT

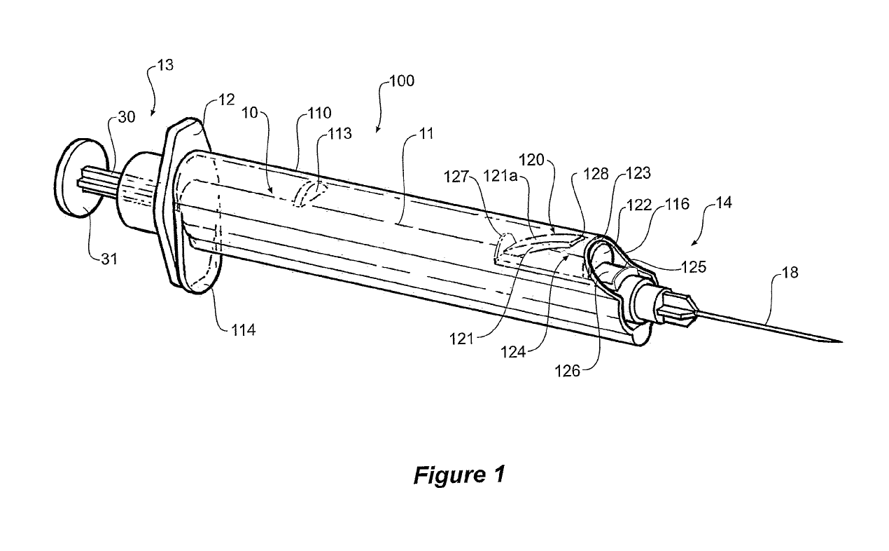

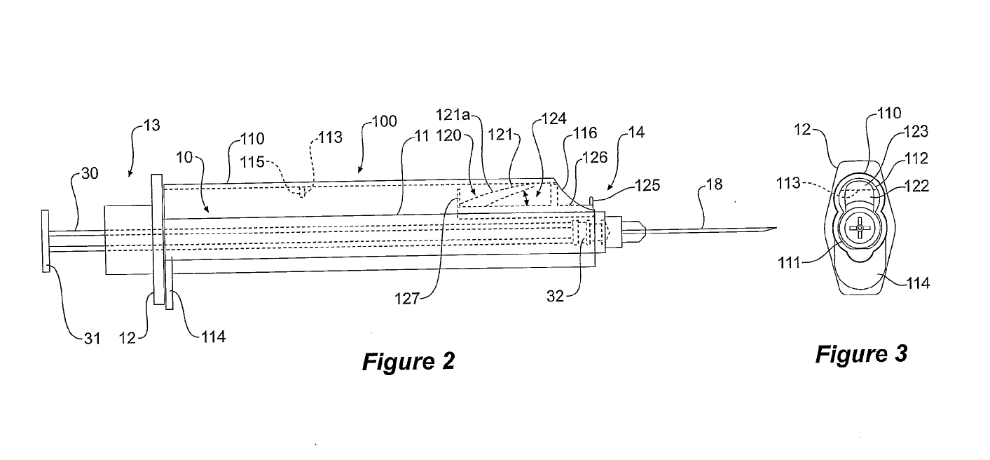

Referring now to FIGS. 1 to 3, there are shown front perspective, side and end on views respectively of a lockable shield assembly 100 according to an preferred embodiment of the present invention. Lockable shield assembly 100 is applied to a standard syringe 10 as is known in the art having a syringe body or barrel 11, a plunger bulb 32 and a plunger arm 30 terminating in a plunger flange 31, the plunger arm 30 adapted to move in the syringe body 11 and a hypodermic needle 18 extending from the syringe body 11. Syringe body 11 includes a shoulder region 12 located at the plunger end 13 of the syringe body 11 remote from hypodermic needle 18.

Lockable shield assembly 100 includes a saddle member 120 which in this preferred embodiment is attached to the needle end 14 of syringe body 11 and a moveable shield or sleeve 110 having two part-cylindrical opposed longitudinal connected inner receiving channels 111, 112 having a figure eight cross-sectional profile (as best seen in FIG. 3). First inner receiving channel 111 is adapted to receive syringe body 11 and second inner receiving channel 112 provides a cavity in which saddle member 120 is received, thereby allowing shield 110 to slide longitudinally along syringe body 11.

An inwardly extending abutment region or projection tab 113 is located on the upper inner surface of second inner receiving channel 112 approximately two thirds along the shield 110 towards the plunger end 13 of syringe body 11. At the plunger end 13 of shield 100 there is located a laterally extending flange 114 which overlays substantially one side of shoulder region 12 of syringe body 11. In another embodiment, a simple circumferential flange is employed. At the needle end 14 of shield 110 there is located a cutaway or access region 116 that partially exposes saddle member 120 with the shield 110 in its retracted position.

Saddle member 120 includes a resilient forward facing prong or tongue 121 extending from the plunger end 13 of saddle member 120 to the needle end 14 of syringe body 11 forming an upwardly sloped ramp region 121a. Tongue 121 is resiliently biased outwardly from syringe body 11. Saddle member 120 also includes an upwardly extending wall portion 122 having a part circular top region 123 which is adapted to conform to the cross-sectional profile of second part-cylindrical inner receiving channel 112. Wall portion 122 is located part way along saddle member 120 forming a catch region 124 between wall portion 122 and the end 128 of tongue 121.

At the front edge or needle end 14 of saddle member 120, there is located a ledge or lip 125 that forms the boundary of front application region 126 of saddle member 120 that extends between wall portion 122 and lip 125. At the rear edge or plunger end 13 of saddle member 120 there is located an upwardly extending flexible tab 127 that extends substantially to the height of end 128 of resilient tongue 121.

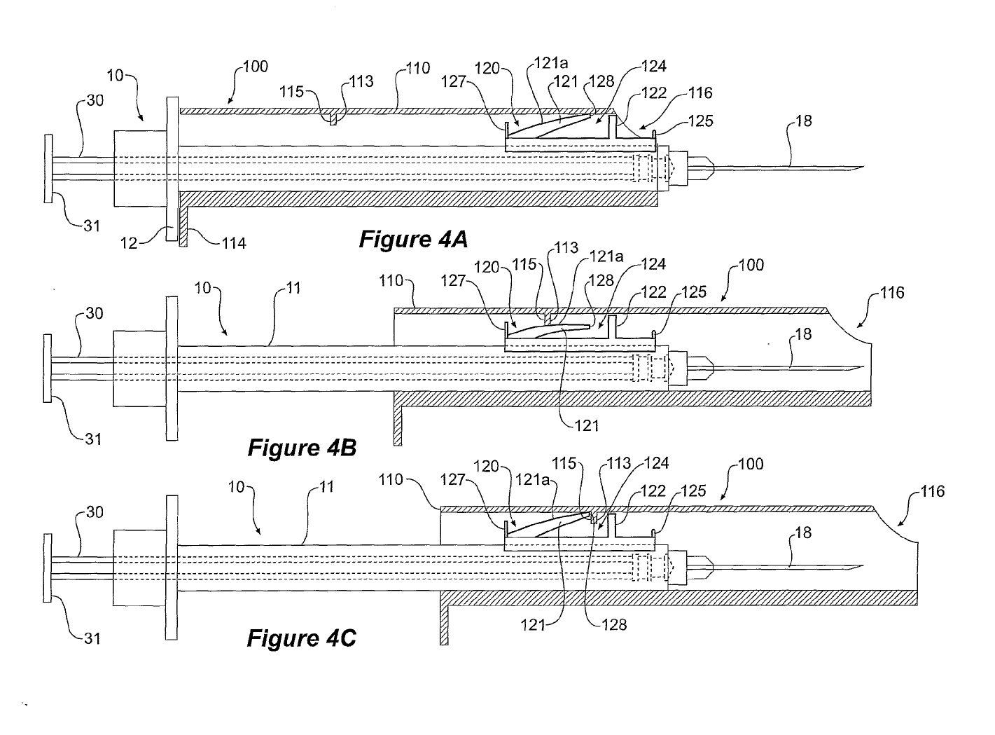

Referring now to FIGS. 4A to 4C, there is shown a series of side views of lockable shield assembly 100 in an initial retracted position (see FIG. 4A), an intermediate position with the shield 110 partially covering needle 18 (see FIG. 4B) and a final fully extended position (FIG. 4C) where shield 110 is locked in position and fully covers needle 18. In the initial retracted position, shield 110 abuts against shoulder region 12. When the syringe is being used the shield 110 will be held against shoulder region 12 by the syringe operator's fingers applying force to flange 114, thereby preventing shield 110 from inadvertently moving forwards towards the hypodermic needle 18.

In the intermediate position, the shield 110 is drawn forward and fully covers needle 18. In the process of moving shield 110 towards the intermediate position, flexible tab 127 will engage against projection tab 113 causing flexible tab 127 to flex forward and then flick backwards, thereby causing a “click” which can both be felt and heard by an operator of syringe 10 thereby in this case providing a tactile and audible indication. This indicates to the operator that shield 110 now covers the needle 18 of syringe 10. As would be apparent to those skilled in the art, other indication means which provide a tactile and/or audible indication to an operator of syringe 10 are contemplated to be within the scope of the present invention.

Further movement of shield 110 causes projection tab 113 to ride along ramp region 121a of resilient tongue 121 causing it to flex towards syringe body 11. In this intermediate position, the frictional engagement between ramp region 121a and projection tab 113 will provide resistance to movement of the shield 110 with more resistance being provided in the forward direction, thereby functioning to at least partially hold shield 110 in place in the intermediate position (as best seen in FIG. 4B). In this manner, the syringe operator may temporarily cover needle 18 with shield 110 should this be required and then retract shield 110 into the retracted position (see FIG. 4A) in the process once again causing a “click” from the interaction of the flexible tab 127 and projection tab 113 to then further continue using syringe 10 as desired.

Once the contents of syringe 10 have been injected, the shield 110 can be extended fully forward causing the projecting tab 113 to ride over ramp region 121a of tongue 121 resulting in the end 128 of tongue 121 flexing back and resting against the shield 110 capturing projecting tab 113 in catch region 124 between the end 128 of tongue and wall portion 122 (as best seen in FIG. 4C). Any attempt to force shield 110 back towards the retracted position will result in the abutment region 115 of projection tab 113 being forced against the end 128 of tongue 121 thereby preventing any backward movement of shield 110. Further forward movement of shield 110 towards the needle end 114 will also be prevented by wall portion 122 resulting in shield 110 being locked in position in the fully extended position and safely covering needle 18. As would be apparent to those skilled in the art, projection tab 113 may be shaped to incorporate a recess or aperture to further capture the end 128 of resilient tongue 121.

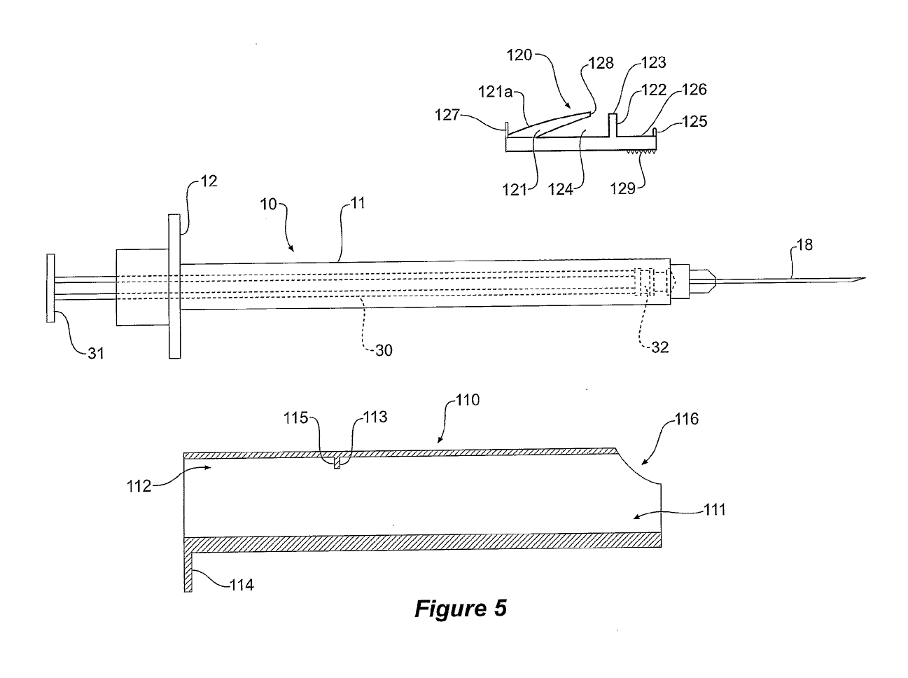

Referring now to FIG. 5, there is shown a disassembled view of the saddle member 120 and sleeve 110 which together form lockable shield assembly 100 for syringe 10. Located under front application region 126 on saddle member 120 is attachment region 129 formed as a series of ridges.

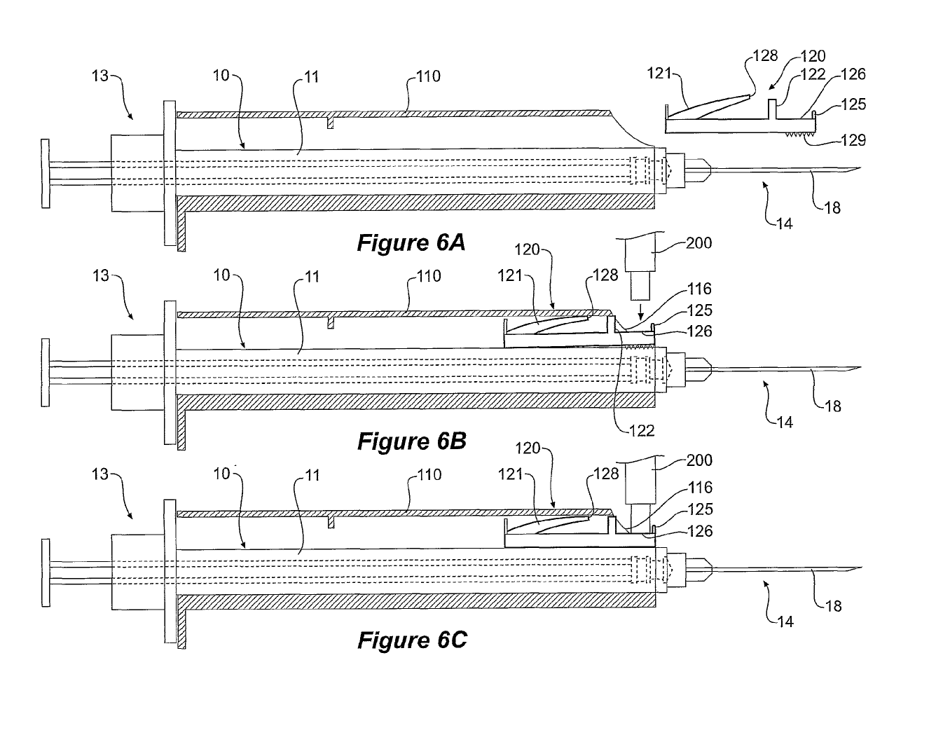

Referring now to FIGS. 6A to 6C, there are shown the various stages of assembling or forming lockable shield assembly 100. These stages are shown figuratively but as would be apparent to those skilled in the art, the features of the present invention make it readily suitable to be adapted to an automated assembly process, thereby substantially reducing manufacturing costs.

At FIG. 6A, syringe 10 is first inserted into lower part-cylindrical inner receiving channel 111 of sleeve 110. The shape of inner receiving channel 111 functions to locate or key onto the cylindrical shape of syringe body 11. When syringe 10 is fully inserted into sleeve 110, cutaway region 116 of sleeve 110 exposes the front end of syringe body 11.

At FIG. 6B, saddle member 120 is inserted into the open end of sleeve 110 into upper part-cylindrical receiving channel 111 of sleeve 110. Attachment region 129 is aligned with the end of syringe body 11. In the process of inserting saddle member 120 into channel 111, resilient tongue 121 will flex inwardly as it moves within sleeve 110 resulting in the end 128 of tongue 121 frictionally engaging with the inner surface of channel 111, thereby functioning to both locate saddle member 120 with respect to sleeve 110 and also helping to prevent movement of sleeve 110 with respect to syringe 10.

Whilst in this embodiment, the cutaway region 116 is located at the needle end 14 of sleeve 110, equally it could be implemented as an access region that is located at other locations on the sleeve. In one example, this access region may form an access aperture located along sleeve 110 which allows attachment of saddle member 120 at a corresponding location on syringe body 11.

As would be apparent to those skilled in the art, this ability to locate the components of lockable shield assembly 100 prior to permanent attachment of saddle member 120 to syringe body 11 greatly facilitates the automation of the assembly process as the individual components to not require separate locating fixtures to ensure that they remain in the correct location, prior to assembly.

In this preferred embodiment, an ultrasonic welder 200 is employed to secure saddle member 120 to syringe body 11. An example of an ultrasonic welder suitable for this task is the electronically controlled Dukane 2120.

As has already been described, saddle member 120 is aligned at the end of syringe body 11 with cutaway region 116 thereby allowing access to application region 126 to receive ultrasonic welder 200 between lip 125 and wall portion 122 which ultrasonically welds attachment region 129 to syringe body 11 as shown in FIG. 6C. This ultrasonic welding process requires less than 0.75 seconds. As would be apparent to those skilled in the art, other attachment methods such as the use of suitable glues are contemplated to be within the scope of the invention.

The attachment process described above provides a bond between saddle member 120 and syringe body 11 that is able to withstand up to approximately 10 kg of force, thereby ensuring that the shield will remain in the locked position and cannot be released due to failure of this bond.

As would be readily appreciated by those skilled in the art, the present invention may be readily adapted to an automated system involving a feeder unit that feeds multiple syringes 10 to a welding station for alignment and attachment of respective saddle members 120. In one example of an automated system for assembling the lockable shield assembly 100 of the present invention there is provided a carousel having multiple stations where each of the assembly steps is carried out.

In this illustrative example, a feeder unit delivers four continuous lines of syringes 10 in a magazine arrangement where the individual syringes 10 are suspended and presented in a substantially vertical orientation and fed sequentially to a first station where an actuator picks the four end syringes 10 from the end of each feed line and inserts them into the first inner receiving channels 111 of respective sleeves 110 which are similarly delivered in a continuous feed line. The partially assembled lockable shield assemblies are then retained in a holder unit which locates and holds four syringes 10 at a time.

The carousel then rotates to a second quality inspection station where each of the partially assembled shield assemblies in the holder unit are inspected by a camera to ensure that the cutaway region 116 of the sleeve 110 is correctly orientated with the end of the syringe body 11. The carousel then rotates to a third station where the holder unit is rotated upwardly through 90 degrees, thereby presenting each of the four syringes 10 in a substantially horizontal orientation to receive and attach the saddle member 120.

In this example, a first pair of saddle members 120 is attached at a fourth station and a second pair of saddle members 120 is attached at a fifth station. In this process, respective saddle members 120 are inserted into the second inner receiving channel 112 of the sleeve 110 by an actuator which positions the saddle member in line with the cutaway or access region 116 on the end of the sleeve 110 at which point the ultrasonic welder 200 attaches the saddle member 120 to the syringe body 11 by application of the welder 200 to the front application region 126 (see also FIGS. 6B and 6C).

The carousel then rotates to a sixth station where each of the ultrasonic welds is stress tested to ensure that there has been adequate bonding between the saddle member 120 and the syringe body 111. After this testing stage, the carousel rotates to a seventh station where the holder unit is rotated back through 90 degrees, thereby presenting the four syringes 10 in a substantially vertical orientation where they are dispensed to be received by a single continuous output feed line for packaging.

Machine vision techniques are used throughout the automated system to ensure the correct orientation of both the component parts prior to assembly and also the quality of the finally assembled product. As would be appreciated by those skilled in the art, the use of these techniques allows for the rapid detection and correction of any defects in the assembly process.

An automated system such as described above is able to assemble 5,000 lockable shield assemblies per hour thus demonstrating a significant advance over prior art systems. Another advantage of this automated system is that only minimal factory floor space is required.

Although a preferred embodiment of the present invention has been described in the foregoing detailed description, it will be understood that the invention is not limited to the embodiment disclosed, but is capable of numerous rearrangements, modifications and substitutions without departing from the scope of the invention as set forth and defined by the following claims.

1. A lockable shield assembly for a syringe, the lockable shield assembly including:
a moveable sleeve adapted to slide longitudinally along a body of the syringe from a retracted position wherein a needle of the syringe is exposed to an extended position wherein the moveable sleeve covers the needle of the syringe; and
a saddle member for attachment to the body of the syringe, the saddle member including a resilient tongue biased outwardly from the saddle member, wherein the resilient tongue in use engages with an abutment region located on an inner surface of the moveable sleeve to prevent retraction of the moveable sleeve when it has been moved to the extended position.

2. The lockable shield assembly of claim 1, wherein the moveable sleeve is operable to move to an intermediate position where the sleeve covers the needle of the syringe but is still able to be retracted to expose the needle of the syringe, and wherein the lockable shield assembly further includes indication means to provide a tactile or audible indication to an operator of the lockable shield assembly that the moveable sleeve is in the intermediate position.

3. The lockable shield assembly of claim 2, wherein the indication means forms part of the saddle member.

4. The lockable shield assembly of claim 3, wherein the indication means includes a flexible member that interacts with the abutment region as the abutment region rides over the flexible member to provide the tactile or audible indication.

5. The lockable shield assembly of claim 4, wherein the flexible member is a flexible tab extending upwardly from the saddle member.

6. The lockable shield assembly of claim 5, wherein the resilient tongue faces forwards towards the needle end of the syringe and wherein the flexible tab is located at the base of the resilient tongue.

7. The lockable shield assembly of claim 1, wherein the saddle member includes an abutment portion operable in combination with the resilient tongue to form a catch region to capture the abutment region of the moveable sleeve when the moveable sleeve is in the extended position.

8. An assembly method for a lockable shield assembly for a syringe, the lockable shield assembly including a moveable sleeve operable to be moved to an extended position by sliding the moveable sleeve along a body of the syringe and a saddle member including a resilient tongue operable to engage with an abutment region located on the moveable sleeve to prevent retraction of the moveable sleeve once it has been moved to the extended position, the method including the steps of:
inserting the body of the syringe into a first receiving channel formed in the moveable sleeve;
inserting the saddle member into a second receiving channel formed in the moveable sleeve, the second receiving channel connected to the first receiving channel; and
attaching the saddle member to the body of the syringe.

9. The assembly method of claim 8, wherein the moveable sleeve includes an access region to allow attachment of the saddle member to the body of the syringe.

10. The assembly method of claim 9, wherein the access region is located at a needle end of the moveable sleeve and is formed as a cutaway region.

11. The assembly method of claim 8, wherein the resilient tongue frictionally engages with an inner surface of the second receiving channel to resist movement of the saddle member with respect to the saddle member prior to assembly.

12. The assembly method of claim 8, wherein the saddle member is attached to the body of the syringe by ultrasonic welding.

13. A saddle member for a lockable shield assembly for a syringe, the saddle member including:
an attachment region for attachment to a body of the syringe; and
a resilient tongue extending outwardly from the saddle member, the resilient tongue operable to engage with an abutment region located on a moveable sleeve of the lockable shield assembly to prevent retraction of the moveable sleeve once it has been moved to an extended position.

14. The saddle member of claim 13, wherein the resilient tongue is adapted to frictionally engage with an inner surface of the moveable sleeve to locate the saddle member on insertion of the saddle member into the sleeve for attachment of the saddle member to the body of the syringe on assembly of the lockable shield assembly.

15. The saddle member of claim 13, wherein the saddle member further includes indication means to provide a tactile or audible indication to an operator of the lockable shield assembly that the moveable sleeve is in an intermediate position where the sleeve covers the needle of the syringe but is still able to be retracted to expose the needle of the syringe.

16. The saddle member of claim 15, wherein the indication means includes a flexible member that interacts with the abutment region as the abutment region rides over the flexible member to provide the tactile or audible indication.

17. The saddle member of claim 16, wherein the flexible member is a flexible tab extending upwardly from the saddle member.

18. The saddle member of claim 17, wherein the resilient tongue faces forwards towards the needle end of the syringe and wherein the flexible tab is located at the base of the resilient tongue.

19. The saddle member of claim 13, wherein the saddle member includes an abutment portion operable in combination with the resilient tongue to form a catch region to capture the abutment region of the moveable sleeve when the moveable sleeve is in the extended position.

20. A moveable sleeve for a lockable shield assembly for a syringe, the moveable sleeve adapted to slide longitudinally along a body of the syringe from a retracted position wherein a needle of the syringe is exposed to an extended position wherein the sleeve covers the needle of the syringe, the moveable sleeve further including an abutment region operable to engage with a resilient tongue extending from a saddle member of the lockable shield assembly to prevent retraction of the moveable sleeve once it has been moved to an extended position.

21. The moveable sleeve of claim 20, wherein the moveable sleeve further includes an access region to allow attachment of the saddle member to the body of the syringe.

22. The moveable sleeve of claim 21, wherein the access region is located at a needle end of the moveable sleeve and is formed as a cutaway region.
